(19) United States
(12) Patent Application Publication
(10) Pub. No.: US 2008/0193530 A1
(43) Pub. Date: Aug. 14, 2008

(54) USE OF RANOLAZINE FOR THE TREATMENT OF NON-CORONARY MICROVASCULAR DISEASES

(76) Inventors: Brent Blackburn, Los Altos, CA (US); Luiz Belardinelli, Palo Alto, CA (US); Andrew Wolff, San Francisco, CA (US)

Correspondence Address: CV THERAPEUTICS, INC., 3172 PORTER DRIVE, PALO ALTO, CA, 94304, US

(21) Appl. No.: 12/030,699

(22) Filed: Feb. 13, 2008

Related U.S. Application Data
(60) Provisional application No. 60/889,734, filed on Feb. 13, 2007.
(60) Provisional application No. 60/893,121, filed on Mar. 5, 2007.
(60) Provisional application No. 60/894,903, filed on Mar. 14, 2007.
(60) Provisional application No. 60/914,645, filed on Apr. 27, 2007.
(60) Provisional application No. 60/941,219, filed on May 31, 2007.
(60) Provisional application No. 60/947,613, filed on Jul. 2, 2007.
(60) Provisional application No. 60/896,478, filed on Mar. 22, 2007.

(51) Int. Cl. A61K 9/22; A61K 31/495; A61P 9/00

(52) U.S. Cl. 424/468; 514/252.12

(57) ABSTRACT

Disclosed are methods for treating patients suffering from non-coronary microvascular disease comprising administering ranolazine to the patient. In one embodiment, ranolazine is administered as an oral dose.

This invention claims priority to U.S. Provisional Patent Application Ser. No. 60/889,734, filed Feb. 13, 2007, U.S. Provisional Patent Application Ser. No. 60/893,121, filed Mar. 5, 2007. U.S. Provisional Patent Application Ser. No. 60/894,903, filed Mar. 14, 2007, U.S. Provisional Patent Application Ser. No. 60/914,645, filed Apr. 27, 2007, U.S. Provisional Patent Application Ser. No. 60/941,219, filed May 31, 2007, U.S. Provisional Patent Application Ser. No. 60/947,613, filed Jul. 2, 2007, and U.S. Provisional Patent Application Ser. No. 60/896,478, filed Mar. 22, 2007, the entireties of each of which is incorporated herein by reference.

### FIELD OF THE INVENTION

This invention relates to methods for treating patients suffering, or at a risk of suffering from, non-coronary microvascular disease.

### DESCRIPTION OF THE ART

U.S. Pat. No. 4,567,264, the specification of which is incorporated herein by reference in its entirety, discloses ranolazine, (±)-N-(2,6-dimethylphenyl)-4-[2-hydroxy-3-(2-methoxyphenoxy)-propyl]-1-piperazineacetamide, and its pharmaceutically acceptable salts, and their use in the treatment of cardiovascular diseases, including arrhythmias, variant and exercise-induced angina, and myocardial infarction. In its dihydrochloride salt form, ranolazine is represented by the formula:

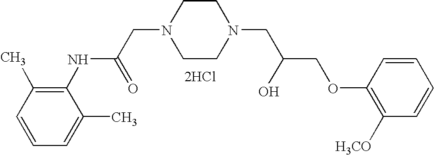

This patent also discloses intravenous (IV) formulations of dihydrochloride ranolazine further comprising propylene glycol, polyethylene glycol 400, Tween 80 and 0.9% saline.

U.S. Pat. No. 5,506,229, which is incorporated herein by reference in its entirety, discloses the use of ranolazine and its pharmaceutically acceptable salts and esters for the treatment of tissues experiencing a physical or chemical insult, including cardioplegia, hypoxic or reperfusion injury to cardiac or skeletal muscle or brain tissue, and for use in transplants. Oral and parenteral formulations are disclosed, including controlled release formulations. In particular, Example 7D of U.S. Pat. No. 5,506,229 describes a controlled release formulation in capsule form comprising microspheres of ranolazine and microcrystalline cellulose coated with release controlling polymers. This patent also discloses IV ranolazine formulations which at the low end comprise 5 mg ranolazine per milliliter of an IV solution containing about 5% by weight dextrose. And at the high end, there is disclosed an IV solution containing 200 mg ranolazine per milliliter of an IV solution containing about 4% by weight dextrose.

The presently preferred route of administration for ranolazine and its pharmaceutically acceptable salts and esters is oral. A typical oral dosage form is a compressed tablet, a hard gelatin capsule filled with a powder mix or granulate, or a soft gelatin capsule (softgel) filled with a solution or suspension. U.S. Pat. No. 5,472,707, the specification of which is incorporated herein by reference in its entirety, discloses a high-dose oral formulation employing supercooled liquid ranolazine as a fill solution for a hard gelatin capsule or softgel.

U.S. Pat. No. 6,503,911, the specification of which is incorporated herein by reference in its entirety, discloses sustained release formulations that overcome the problem of affording a satisfactory plasma level of ranolazine while the formulation travels through both an acidic environment in the stomach and a more basic environment through the intestine, and has proven to be very effective in providing the plasma levels that are necessary for the treatment of angina and other cardiovascular diseases.

U.S. Pat. No. 6,852,724, the specification of which is incorporated herein by reference in its entirety, discloses methods of treating cardiovascular diseases, including arrhythmias variant and exercise-induced angina and myocardial infarction.

U.S. Patent Application Publication Number 2006/0177502, the specification of which is incorporated herein by reference in its entirety, discloses oral sustained release dosage forms in which the ranolazine is present in 35-50%, preferably 40-45% ranolazine. In one embodiment the ranolazine sustained release formulations of the invention include a pH dependent binder; a pH independent binder; and one or more pharmaceutically acceptable excipients. Suitable pH dependent binders include, but are not limited to, a methacrylic acid copolymer, for example Eudragit® (Eudragit® L100-55, pseudolatex of Eudragit® L100-55, and the like) partially neutralized with a strong base, for example, sodium hydroxide, potassium hydroxide, or ammonium hydroxide, in a quantity sufficient to neutralize the methacrylic acid copolymer to an extent of about 1-20%, for example about 3-6%. Suitable pH independent binders include, but are not limited to, hydroxypropylmethylcellulose (HPMC), for example Methocel® E10M Premium CR grade HPMC or Methocel® E4M Premium HPMC. Suitable pharmaceutically acceptable excipients include magnesium stearate and microcrystalline cellulose (Avicel® pH101).

Non-coronary microvascular disease is a disease of the peripheral small blood vessel including the small blood vessels of the eye, the kidney and/or of the sheaths around the nerves etc. In microvascular disease, the small vessels can lose their ability to dilate and increase blood flow to the heart. The cause is not fatty deposits like the ones that can block the coronary arteries. Rather, the muscles in the arterioles thicken, a process called remodeling, and the walls may stiffen and begin to close in. The result can be ischemia, lack of blood flow.

Microvascular complications associated with diabetes mellitus in mammals are the microvascular diseases associated with the vascular system of the retina, kidney, nerves, skin and brain (Tsilibary E. C. The Journal of Pathology 2003, 200 (4):537-546). The chronic hyperglycemia of diabetes is associated with long-term damage, dysfunction, and failure of various organs, especially the eyes, kidneys, nerves, heart, and blood vessels and long-term complications of diabetes include retinopathy with potential loss of vision; nephropathy leading to renal failure; peripheral neuropathy with risk of foot ulcers, amputation, and Charcot joints; and autonomic neuropathy causing gastrointestinal, genitourinary, cardiovascular symptoms and sexual dysfunction.

Another type of disease associated with microvascular disease is cerebrovascular disease, including transient ischemic attacks, which can be described as brief episodes of neurological dysfunction caused by focal brain or retinal ischemia, with clinical symptoms typically lasting less than one hour, and without evidence of acute infarction (see Solenski, N. J., Am. Fam. Phys. (2004) 69 (9) rec. No: 1665). Microvascular disease has been found to alter brain activation in Type 1 diabetic patients (see Wessels, A. M. et al. Diabetes, 2006, 55:334-340). Various forms of cerebrovascular insufficiencies such as, reduced blood supply to the brain or disrupted microvascular integrity in cortical regions may result in cognitive failure and result in neurodegenerative processes such as aging and Alzheimer's disease.

Another type of disease associated with microvascular disease is Restless Legs Syndrome (RLS), a neurological disorder characterized by unpleasant sensations in the legs and an uncontrollable urge to move when at rest in an effort to relieve these feelings.

Microvascular disease can also be associated with dysfunction of the major organs, including the eyes, nerves, skin, brain, kidney, liver, and lungs. Microvascular disease of the major organs can be associated with the presence hypertension, incipient diabetes, and/or metabolic syndrome.

Treatment of microvascular diseases include, diet, exercise, treating mental stress and depression, treating low levels of estrogen before menopause, reducing lipid abnormalities such as low high density lipoprotein (HDL), high low density lipoprotein (LDL), and high triglycerides), and treating hypertension. Patient suffering from microvascular disease often use medications to control any underlying risk factors, such as high blood pressure, high cholesterol and glucose intolerance. These treatments albeit helpful, are often not sufficient in effective treatment of microvascular diseases. Accordingly, a need exists for improved methods and compositions for treating microvascular diseases.

### SUMMARY OF THE INVENTION

This invention relates to a method for treating a patient suffering from a non-coronary microvascular disease. One aspect of the invention provides for a method for treating a patient suffering from non-coronary microvascular disease comprising selecting a patient suffering from, or at risk of suffering from, non-coronary microvascular disease, and administering to that patient an effective amount of ranolazine.

In one embodiment, the patient is not suffering from diabetes. In other embodiments, the patient is suffering from at least one condition selected from the group consisting of hypertension, incipient diabetes, and metabolic syndrome.

The non-coronary microvascular disease can manifest itself in the eye, the brain, the kidney, and the liver.

In a preferred embodiment, the ranolazine is administered to the patient as an oral dose, preferably a sustained release tablet.

### DESCRIPTION OF THE FIGURES

FIGS. 1 and 2 illustrate the HbA1C serum levels in both non-diabetic and diabetic patients who were treated with ranolazine.

### DETAILED DESCRIPTION OF THE INVENTION

As noted above, this invention relates to methods for treating patients suffering from microvascular diseases comprising administering ranolazine to these patients. However, prior to describing this invention in more detail, the following terms will first be defined.

### Definitions

As noted above, this invention relates to methods for treating patients suffering from microvascular diseases comprising administering ranolazine to these patients. However, prior to describing this invention in more detail, the following terms will first be defined.

### Definitions

In this specification and in the claims that follow, reference will be made to a number of terms that shall be defined to have the following meanings.

“Ranolazine” is the compound (±)-N-(2,6-dimethylphenyl)-4-[2-hydroxy-3-(2-methoxyphenoxy)propyl]-1-piperazine-acetamide, and its pharmaceutically acceptable salts, and mixtures thereof. Unless otherwise stated the ranolazine plasma concentrations used in the specification and examples refer to ranolazine free base. At pH ˜4, in an aqueous solution titrated with hydrogen chloride, ranolazine will be present in large part as its dihydrochloride salt.

“Physiologically acceptable pH” refers to the pH of an intravenous solution which is compatible for delivery into a human patient. Preferably, physiologically acceptable pH's range from about 4 to about 8.5 and preferably from about 4 to 7. Without being limited by any theory, the use of intravenous solutions having a pH of about 4 to 6 are deemed physiologically acceptable as the large volume of blood in the body effectively buffers these intravenous solutions.

“Coronary diseases” or “cardiovascular diseases” refer to diseases of the cardiovasculature arising from atherosclerosis, thombosis, myocardial infarction, and/or ischemia (including recurrent ischemia) of the coronary vasculature as well as macroscopic coronary dysfunction such as, heart failure (including congestive heart failure, acute heart failure) and intermittent claudication. A symptom of one or more of these coronary diseases may include angina, such as exercise-induced angina, variant angina, stable angina and unstable angina.

The treatment of such disease states is disclosed in various U.S. patents and patent applications, including U.S. Pat. Nos. 6,503,911 and 6,528,511, U.S. Patent Application Serial Nos. 2003/0220344 and 2004/0063717, the complete disclosures of which are hereby incorporated by reference.

“Non-coronary microvascular disease” refers to a disease attributable to the degeneration of the microvascular wherein the disease is not a coronary disease.

Without being limited to any theory, non-coronary microvascular disease is believed to be associated with one or a combination of the following risk factors: high cholesterol, high blood pressure, hyperlipidemia, incipient diabetes, metabolic syndrome, smoking, and damage to the delicate lining of the small arteries from chemicals present in the blood, causing the blood to clot in the artery.

“Metabolic syndrome” refers to a disorder characterized by a group of metabolic risk factors present in one person. The metabolic risk factors include central obesity (excessive fat tissue in and around the abdomen), atherogenic dyslipidemia (blood fat disorders—mainly high triglycerides and low HDL cholesterol), insulin resistance or glucose intolerance, prothrombotic state (e.g., high fibrinogen or plasminogen activator inhibitor in the blood), and high blood pressure (130/85 mmHg or higher).

Metabolic syndrome, in general, can be diagnosed based on the presence of three or more of the following clinical manifestations in one subject:
a) Abdominal obesity characterized by a elevated waist circumference equal to or greater than 40 inches (102 cm) in men and equal to or greater than 35 inches (88 cm) in women;
b) Elevated triglycerides equal to or greater than 150 mg/dL;
c) Reduced levels of high-density lipoproteins of less than 40 mg/dL in women and less than 50 mg/dL in men;
d) High blood pressure equal to or greater than 130/85 mm Hg; and
e) Elevated fasting glucose equal to or greater than 100 mg/dL.

“Incipient diabetes” refers to a state where a subject has elevated levels of glucose or, alternatively, elevated levels of glycosylated hemoglobin such as HbA1c, but has not developed diabetes.

“Optional” and “optionally” mean that the subsequently described event or circumstance may or may not occur, and that the description includes instances where the event or circumstance occurs and instances in which it does not. For example, “optional pharmaceutical excipients” indicates that a formulation so described may or may not include pharmaceutical excipients other than those specifically stated to be present, and that the formulation so described includes instances in which the optional excipients are present and instances in which they are not.

“Treating” and “treatment” refer to any treatment of a disease in a patient and include: preventing the disease from occurring in a subject which may be predisposed to the disease but has not yet been diagnosed as having it; inhibiting the disease, i.e., arresting its further development; inhibiting the symptoms of the disease; relieving the disease, i.e., causing regression of the disease, or relieving the symptoms of the disease. The “patient” is a mammal, preferably a human.

“Immediate release” (“IR”) refers to formulations or dosage units that rapidly dissolve in vitro and are intended to be completely dissolved and absorbed in the stomach or upper gastrointestinal tract. Conventionally, such formulations release at least 90% of the active ingredient within 30 minutes of administration.

“Sustained release” (“SR”) refers to formulations or dosage units used herein that are slowly and continuously dissolved and absorbed in the stomach and gastrointestinal tract over a period of about six hours or more. Preferred sustained release formulations are those exhibiting plasma concentrations of ranolazine suitable for no more than twice daily administration with two or less tablets per dosing as described below.

“Intravenous (IV) infusion” or “intravenous administration” refers to solutions or dosage units used herein that are provided to the patient by intravenous route. Such IV infusions can be provided to the patient until for up to about 96 hours in order to stabilize the patient's cardiovascular condition. The method and timing for delivery of an IV infusion is within the skill of the attending medically trained person.

“Renal insufficiency” refers to when a patient's kidneys no longer have enough kidney function to maintain a normal state of health. Renal insufficiency includes both acute and chronic renal failure, including end-stage renal disease (ESRD).

“Patients at risk of non-coronary microvascular disease” refers to those patients having been diagnosed with incipient diabetes, metabolic syndrome, etc. but which diseases have yet to manifest symptoms such as peripheral neuropathy.

Methods of this Invention

In one aspect, this invention provides for a method for treating a patient suffering from non-coronary microvascular disease by administering ranolazine. In a preferred embodiment, a patient is selected for treatment with ranolazine by determining if that patient is suffering from, or is at a risk of suffering from, non-coronary microvascular disease prior to the administration of ranolazine. In a further preferred embodiment, the patient is not suffering from diabetes.

Methods of determining if a patient is suffering from, or is likely to be suffering from, non-coronary microvascular disease include assessment of risk factors determined to be associated with the presence of coronary microvascular disease. Risk factors to be assessed include determining if the patient is suffering from metabolic syndrome, hypertension, and/or incipient diabetes.

Accordingly, one embodiment of the invention provides for a method for treating a patient suffering from non-coronary microvascular disease comprising the steps of selecting a patient suffering from, or likely to be suffering from, non-coronary microvascular disease and administering to that patient an effective amount of ranolazine.

The non-coronary microvascular disease can manifest itself any number of organs including eye, kidney, liver, lungs, nerves, skin, and brain. It can also manifest itself as a disorder such as Legs Syndrome (RLS).

Long term care of patients suffering from microvascular diseases by the methods described herein are preferably achieved by administering to the presenting patient an effective amount of an oral dose of ranolazine. Oral dosing is dependent upon the condition of the patient, the age, weight, and otherwise general health of the patient. Such factors are well within the skill of the attending clinician and are discussed in detail below.

Alternatively, patients presenting with acute symptoms of microvascular disease can be initially treated with an effective amount of an IV dose of ranolazine in order to rapidly attain a therapeutic serum level. IV dosing is dependent upon the condition of the patient, the age, weight, and otherwise general health of the patient. Such factors are well within the skill of the attending clinician and are discussed in detail below.

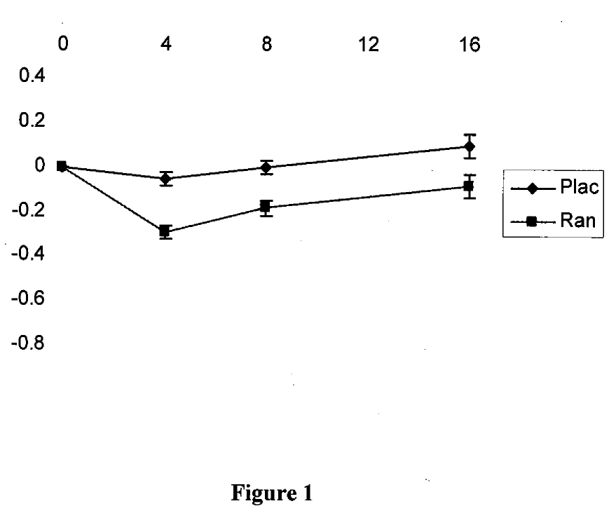

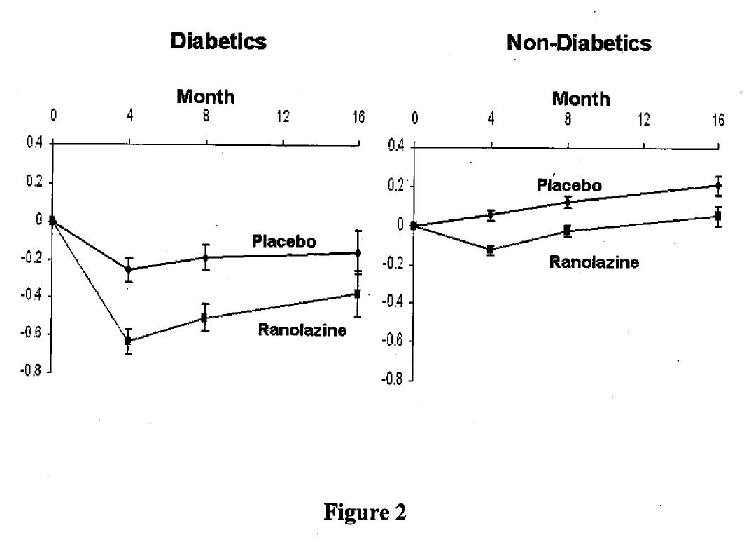

Incipient diabetic patients at risk of non-coronary microvascular disease are benefited by treatment with ranolazine as per this invention because ranolazine significantly lowers serum HbA1C levels as shown in FIGS. 1 and 2. Such lowering of the HbA1C levels reduces the likelihood of the incipient diabetes further degrading into diabetes itself and prevents the further progression of microvascular diseases.

Compositions of the Invention

Oral Dose

In one embodiment, the ranolazine is administered an oral dose. In one embodiment, an oral formulation of ranolazine is a tablet. The tablet can be formulated as an instant release or a sustained release tablet. In one embodiment, the tablet of ranolazine is up to 750 mg ranolazine. In a preferred embodiment, the ranolazine tablet is 375 mg ranolazine, and/or 500 mg ranolazine.

The oral formulation of ranolazine is thoroughly discussed in U.S. Pat. No. 6,303,607 and U.S. Publication No. 2003/0220344, which are both incorporated herein by reference in their entirety.

The oral sustained release ranolazine dosage formulations of this invention are administered one, twice, or three times in a 24 hour period in order to maintain a plasma ranolazine level above the threshold therapeutic level and below the maximally tolerated levels, which is preferably a plasma level of about 550 to 7500 ng base/mL in a patient.

In a preferred embodiment, the plasma level of ranolazine ranges about 1500-3500 ng base/mL.

In order to achieve the preferred plasma ranolazine level, it is preferred that the oral ranolazine dosage forms described herein are administered once or twice daily. If the dosage forms are administered twice daily, then it is preferred that the oral ranolazine dosage forms are administered at about twelve hour intervals.

In addition to formulating and administering oral sustained release dosage forms of this invention in a manner that controls the plasma ranolazine levels, it is also important to minimize the difference between peak and trough plasma ranolazine levels. The peak plasma ranolazine levels are typically achieved at from about 30 minutes to eight hours or more after initially ingesting the dosage form while trough plasma ranolazine levels are achieved at about the time of ingestion of the next scheduled dosage form. It is preferred that the sustained release dosage forms of this invention are administered in a manner that allows for a peak ranolazine level no more than 8 times greater than the trough ranolazine level, preferably no more than 4 times greater than the trough ranolazine level, preferably no more than 3 times greater than the trough ranolazine level, and most preferably no greater than 2 times trough ranolazine level.

The sustained release ranolazine formulations of this invention provide the therapeutic advantage of minimizing variations in ranolazine plasma concentration while permitting, at most, twice-daily administration. The formulation may be administered alone, or (at least initially) in combination with an immediate release formulation if rapid achievement of a therapeutically effective plasma concentration of ranolazine is desired or by soluble IV formulations and oral dosage forms.

Intravenous Dose

In one embodiment, the ranolazine is administered to the presenting patient by an IV solution comprising a selected concentration of ranolazine. Heretofore, the art provided IV solutions comprising ranolazine which comprised low concentrations of ranolazine (see, e.g., Kluge et al., U.S. Pat. No. 4,567,264 where Example 11 of that patent describes using 1.4 mg of ranolazine per mL in an IV solution comprising significant amounts of both propylene glycol (20 g/100 mL) and polyethylene glycol (20 g/100 mL)). Propylene glycol is a viscous liquid as is polyethylene glycol (see, e.g., the Merck Index, 12^{th} Ed., 1996). The increased viscosity resulting from the use of such IV solutions makes the rapid delivery of ranolazine to the patient suffering from an acute cardiovascular disease event more cumbersome and requires that a significant amount of propylene glycol and polyethylene glycol be co-administered.

Alternatively, the art provided IV solutions comprising ranolazine which comprised either high or very high concentrations of ranolazine (either 5 mg/mL or 200 mg/mL) relative to that employed in the IV solutions used herein. See, e.g., Dow, et al., U.S. Pat. No. 5,506,229. In an acute cardiovascular disease event where the patient is suffering from or at risk of suffering from renal insufficiency, the use of such concentrations of ranolazine can result in higher ranolazine plasma levels. Accordingly, the use of such concentrations is contraindicated for treating patients presenting with an acute cardiovascular disease event as the attending physician has little if any time to assess the renal function of that patient prior to initiating treatment.

In the methods of this invention, the IV solution has a selected amount of ranolazine comprising from about 1.5 to 3 mg per milliliter of solution, preferably about 1.8 to 2.2 mg per milliliter and, even more preferably, about 2 mg per milliliter. In contrast to Kluge, et al., supra., the IV solution does not contain any propylene glycol or any polyethylene glycol. Rather the compositions of this invention comprise ranolazine, sterile water and dextrose monohydrate or sodium chloride. As such, the compositions of this invention are less viscous than those described by Kluge et al. allowing for more efficient rapid titration of the patient with the IV solution.

The IV solution of this invention is different from the injectable formulations since injectable formulations typically have excipients that may not be needed and may be contraindicated for IV formulations of this invention. For example, an injectable formulation can have an anti-spasmodic agent such as gluconic acid. As such, the IV solutions of this invention do not contain such anti-spasmodic agents and especially gluconic acid.

The IV solution of this invention is used to stabilize a patient suffering from an acute cardiovascular disease event. In particular, the presenting patient is immediately administered this IV solution of ranolazine for a period until the patient is stabilized. Such stabilization typically occurs within from about 12 to about 96 hours.

In a preferred embodiment, the patient suffering from an acute cardiovascular disease event is treated by:
a) initiating administration of an IV solution to said patient wherein said IV solution comprises a selected concentration of ranolazine of from about 1.5 to about 3 mg per milliliter, preferably about 1.8 to about 2.2 mg per milliliter and, even more preferably, about 2 mg per milliliter;
b) titrating the IV administration of the IV ranolazine solution to the patient comprising: i) a sufficient amount of the IV solution to provide for about 200 mg of ranolazine delivered to the patient over about a 1 hour period; ii) followed by either: a sufficient amount of the IV solution to provide for about 80 mg of ranolazine per hour; or if said patient is suffering from renal insufficiency, a sufficient amount of the IV solution to provide for about 40 mg of ranolazine per hour; and
c) maintaining the titration of b) above until the patient stabilizes which typically occurs within from about 12 to about 96 hours.

In one embodiment, the infusion of the intravenous formulation of ranolazine is initiated such that a target peak ranolazine plasma concentration of about 2500 ng base/mL (wherein ng base/mL refers to ng of the free base of ranolazine/mL) is achieved.

The downward adjustment of ranolazine infusion for a patient experiencing adverse events deemed to be treatment related, is within the knowledge of the skilled in the art and, based on the concentration of ranolazine in the IV solution, easy to achieve. Adverse events in addition to those described above include, but are not limited to, profound and persistent QTc prolongation, not attributed to other reversible factors such as hypokalemia; dizziness; nausea/vomiting; diplopia; parasthesia; confusion; and orthostatic hypotension. In one embodiment, the dose of intravenous solution of ranolazine may be adjusted to a lower dose such as, but not limited to, about 60 mg/hr, about 40 mg/hr, or about 30 mg/hr. In another embodiment, the intravenous delivery of ranolazine may be temporarily discontinued for 1-3 hrs and then restarted at the same or lower dose for patients experiencing adverse events deemed to be treatment related.

In a preferred embodiment, once stabilized the patient is then administered an oral sustained release formulation of ranolazine. In one embodiment, the oral dose of ranolazine is administered about 1 hour prior to the termination of the intravenous infusion of ranolazine. In one aspect of this embodiment, at the time of transition from intravenous to oral dose, for the intravenous dose of ranolazine of about 80 mg/hr, the oral dose administered is 1000 mg once or twice daily (2×500 mg). In another aspect of this embodiment, at the time of transition from intravenous to oral dose, for the intravenous dose of ranolazine of about 60 mg/hr, the oral dose administered is 750 mg once or twice daily (2×375 mg). In still another aspect of this embodiment, at the time of transition from intravenous to oral dose, for the intravenous dose of ranolazine of about 40 mg/hr, the oral dose administered is 500 mg (1×500 mg). In still another aspect of this embodiment, at the time of transition from intravenous to oral dose, for the intravenous dose of ranolazine of about 30 mg/hr, the oral dose administered is 375 mg (1×375 mg).

The downward adjustment of the oral dose for a patient experiencing adverse events deemed to be treatment related, is also within the knowledge of the skilled in the art. For example, the oral dose of ranolazine can be adjusted for patients with newly developed severe renal insufficiency. Other adverse events include, but are not limited to, profound and persistent QTc prolongation, not attributed to other reversible factors such as hypokalemia; dizziness; nausea/vomiting; diplopia; parasthesia; confusion; and orthostatic hypotension. In one embodiment, the oral dose of ranolazine may be adjusted downward to 500 mg once or twice daily, if not already at this dose or lower. In one embodiment, the oral dose of ranolazine may be adjusted to the next lower dose such as, but not limited to, 750 mg once or twice daily, 500 mg once or twice daily, or 375 mg once or twice daily.

Combination Therapy

Patients being treated for microvascular disease often exhibit diseases or conditions that benefit from treatment with other therapeutic agents. These diseases or conditions can be of the cardiovascular nature or can be related to pulmonary disorders, metabolic disorders, gastrointestinal disorders and the like. Additionally, some patients being treated for microvascular disease by administration of ranolazine exhibit conditions that can benefit from treatment with therapeutic agents that are antibiotics, analgesics, and/or antidepressants and anti-anxiety agents.

Examples of combination therapy which may be beneficial to a patient suffering from microvascular disease include therapeutic agents suitable for treating cardiovascular related diseases or conditions including anti-anginals, heart failure agents, antithrombotic agents, antiarrhythmic agents, antihypertensive agents, and lipid lowering agents.

The co-administration of ranolazine with therapeutic agents suitable for treating cardiovascular related conditions allows enhancement in the standard of care therapy the patient is currently receiving.

Accordingly, one aspect of the invention provides a method for treating a patient suffering from microvascular disease and at least one other disease or condition, which method comprises administering to the patient ranolazine in combination with at least one therapeutic agent. In an alternative embodiment, the invention provides a method for treating a patient suffering from microvascular disease and at least two other diseases or conditions, the method comprising administering to the patient ranolazine in combination with at least two therapeutic agents.

The methods of combination therapy include coadministration of a single formulation containing the ranolazine and therapeutic agent or agents, essentially contemporaneous administration of more than one formulation comprising the ranolazine and therapeutic agent or agents, and consecutive administration of ranolazine and therapeutic agent or agents, in any order, wherein preferably there is a time period where the ranolazine and therapeutic agent or agents simultaneously exert their therapeutic affect. Preferably the ranolazine is administered in an oral dose as described herein.

The following Examples are representative of the invention, but are not to be construed as limiting the scope of the claims.

### EXAMPLE 1

### Patient Demographics and Selection

Patients at risk of developing non-coronary microvascular disease include patients that have hypertension, incipient diabetes, metabolic syndrome.

The conditions associated with metabolic syndrome include central obesity (excessive fat tissue in and around the abdomen), atherogenic dyslipidemia (blood fat disorders—mainly high triglycerides and low HDL cholesterol), insulin resistance or glucose intolerance, prothrombotic state (e.g., high fibrinogen or plasminogen activator inhibitor in the blood), and high blood pressure (130/85 mmHg or higher). Methods of determining the presence of these conditions are well known in the art.

Incipient diabetes can be determined by measuring the level of HbA1c levels and hypertension is determined by measuring the blood pressure of a patient. Both of these measurements can be performed using methods well known in the art.

### EXAMPLE 2

### Method of Preparing a Sustained Release Tablet

One sustained release formulation of ranolazine employed in this invention, includes a pH dependent binder and a pH independent binder. This formulation was prepared by combining Ranolazine (7500 g), Eudragit(r) L 100-55 (1000 g), hydroxypropyl methylcellulose (Methocel(r) E5-source) (200 g), and microcrystalline cellulose (Avicel(r)) (1060 g) by intimate mixing. The mixed powders were granulated with a solution of sodium hydroxide (40 g) in water (1900 to 2500 g). The granulate was dried and screened, mixed with magnesium stearate (200 g), and compressed for example into tablets weighing 667 mg to achieve a dose of 500 mg of ranolazine free base per tablet. The tablets were spray coated in a 24 inch Accelacota(r) cylindrical pan coater with OPADRY film coating solution to a 2-4% weight gain. OPADRY film coating solutions are available in a variety of colors from Colorcon (West Point, Pa.).

The stepwise procedure for preparing this formulation is as follows:

a) Blend together ranolazine, microcrystalline cellulose, methacrylate copolymer (Type C) and hydroxypropyl methyl cellulose using an appropriate blender.

b) Dissolve sodium hydroxide in purified water.

c) Using appropriate granulation equipment, slowly add the sodium hydroxide solution to the blend with constant mixing. Add a further aliquot of water, if necessary.

d) Continue mixing to achieve additional massing. Add a further aliquot of water, if necessary.

e) Dry granulated in a fluid bed dryer.

f) Screen dried granules through an appropriate mill.

g) Add magnesium stearate to the screened granules and blend together.

h) Pass the granulated material through a chilsonator, if needed.

i) Compress the granules into tablets using appropriately sized tooling.

j) Disperse OPADRY powder in water and film-coat using appropriately sized coating equipment to a typical level of 2-4% by weight.

k) Polish with carnauba wax using a typical level of 0.002-0.003% by weight.

### EXAMPLE 3

### Preparation of IV Ranolazine

20-mL Type 1 flint vial of Ranolazine Injection filled to deliver 20 mL (at 1, 5, or 25 mg/mL ranolazine concentration).

Compositions:

| | |
|---|---|
| Ranolazine | 1.0, 5.0, 25.0 mg/mL |
| Dextrose monohydrate | 55.0, 52.0, 36.0 mg/mL |
| Hydrochloric acid | q.s. pH to 4.0 ± 0.2 |
| Sodium hydroxide | q.s. pH to 4.0 ± 0.2 |
| Water for Injection | q.s. |

Container/Closure System:

| | |
|---|---|
| Vial: | Type 1 Flint, 20-cc, 20-mm finish |
| Stopper: | Rubber, 20-mm, West 4432/50, gray butyl, teflon coated |
| Seal: | Aluminum, 20-mm, flip-top oversea |

### Method of Manufacture

The intraveous formulation of ranolazine is manufactured via an aseptic fill process as follows. In a suitable vessel, the required amount of dextrose monohydrate was dissolved in Water for Injection (WFI) at about 78% of the final batch weight. With continuous stirring, the required amount of ranolazine was added to the dextrose solution. To facilitate the dissolution of ranolazine, the solution pH was adjusted to a target of 3.88-3.92 with an 0.1 N or 1.0 N HCl solution. Additionally, 1 N NaOH may have been utilized to further adjust the solution to the target pH of 3.88-3.92. After ranolazine was dissolved, the batch was adjusted to the final weight with WFI. Upon confirmation that in-process specifications had been met, the ranolazine-formulated bulk solution was sterilized by sterile filtration through two 0.2 μm sterile filters. Subsequently, the sterile ranolazine-formulated bulk solution was aseptically filled into sterile glass vials and aseptically stoppered with sterile stoppers. The stoppered vials were then sealed with clean flip-top aluminum overseals. The vials then went through a final inspection.

### EXAMPLE 4

### Preparation of IV Ranolazine

20-mL Type 1 flint vial of Ranolazine Injection are filled to deliver 20 mL (25 mg/mL concentration).

Composition:

| | |
|---|---|
| Ranolazine | 25.0 mg/mL |
| Dextrose monohydrate | 36.0 mg/mL |
| Hydrochloric acid | Adjust pH to 3.3-4.7 |
| Water for Injection | q.s. |

Container/Closure System:

| | |
|---|---|
| Vial: | Type 1 tubing, untreated, 20-mL, 20-mm finish |
| Stopper: | Rubber, 20-mm, West 4432/50, gray butyl |
| Seal: | Aluminum, 20-mm, blue flip-off overseal |

### Method of Manufacture

Water for Injection (WFI) is charged in a suitable vessel at about 90% of the final batch weight. About 90-95% of the required amount of 5 N HCl is added into the compounding vessel. With continuous stirring, the required amount of ranolazine is slowly added, followed by the addition of dextrose monohydrate into the ranolazine solution. To solubilize ranolazine, the solution pH is adjusted with 5 N HCl solution to a target of 3.9-4.1. The batch is subsequently adjusted to the final weight with WFI. Upon confirmation that in-process specifications have been met, the ranolazine-formulated bulk solution is sterilized by filtration through two redundant 0.22 μm sterilizing filters. The sterile ranolazine-formulated bulk solution is then aseptically filled into 20 mL sterile/depyrogenated vials and aseptically stoppered with sterile/depyrogenated stoppers. The stoppered vials are sealed with clean flip-top aluminum overseals. The sealed vials are terminally sterilized by a validated terminal sterilization cycle at 121.1° C. for 30 minutes. After the terminal sterilization process, the vials go through an inspection. To protect the drug product from light, the vials are individually packaged into carton boxes.

### EXAMPLE 5

### Treatment of Patients Suffering From Non-Coronary Microvascular Disease

A patient is selected as suffering from, or at a risk of suffering from, non-coronary microvascular disease, using the criteria and methods of Example 1. After selection, the patient is administered an oral dose of ranolazine in an amount effective to treat non-coronary microvascular disease.

### EXAMPLE 6

### The Effect of Ranolazine on Patients with Diabetes or no Diabetes

A clinical study was conducted to evaluate the effect of administration of ranolazine in randomized patients having coronary disease a portion of which also had a glycosylated hemoglobin level (HbA1C)≧7% at presentation. The patients were treated with either placebo or ranolazine as shown in Table 1. The patients were administered an IV dose of ranolazine followed by administration of an oral sustained release dose of ranolazine as described herein. It is contemplated that dosing with oral ranolazine alone will have similar results.

Baseline is in-hospital visit prior to hospitalization.

TABLE 1
Proportion of patients with HbA1C ≧7%

| | Placebo | Ranolazine |
|---|---|---|
| | (n = 3273) | (n = 3268) |
| Baseline | 687 (25%) | 655 (24%) |
| Month 4 | 542 (22%) | 404 (17%) |
| Month 8 | 439 (22%) | 335 (18%) |
| Month 16 | 87 (20%) | 84 (20%) |
| Final Visit | 514 (21%) | 416 (17%) |

Table 2 below shows the proportion of patients having a HbA1c levels greater than 7% (diabetic patients) at the start of the clinical study and the corresponding number at certain intervals during the clinical study.

TABLE 2
Proportion of patients with HbA1c ≧7% by diabetes at enrollment

| | Diabetes | | No Diabetes | |
|---|---|---|---|---|
| | Placebo | Ranolazine | Placebo | Ranolazine |
| Visit | (n = 1117) | (n = 1098) | (n = 2156) | (n = 2170) |
| Baseline | 559 (56%) | 542 (57%) | 128 (7%) | 113 (6%) |
| Month 4 | 424 (51%) | 326 (41%) | 118 (7%) | 78 (5%) |
| Month 8 | 339 (52%) | 263 (44%) | 100 (8%) | 72 (6%) |
| Month 16 | 70 (53%) | 63 (48%) | 17 (6%) | 21 (7%) |
| Final Visit | 413 (50%) | 324 (42%) | 101 (6%) | 92 (6%) |

FIGS. 1-2 show that ranolazine has a HbA1c lowering effect on all patients treated. The data suggests that the results may not be a diabetes specific effect. Nevertheless, where a patient requires lowering of his/her HbA1c levels, ranolazine can provide a beneficial result.

Specifically, in FIG. 1, the reduction of HbA1c levels in all patients treated with ranolazine or placebo is provided on the Y axis whereas the X axis measures the time period in months of ranolazine administration.

In FIG. 2, the comparison of the reduction of HbA1c levels in diabetic patients treated with ranolazine is compared to non-diabetic patients treated with ranolazine. In both cases, there is a reduction in the amount of HbA1C levels demonstrating a non-specific event (i.e., ranolazine reduces HbA1C levels independent of whether the patient is or is not diabetic.)

What is claimed is:

1. A method for treating a patient suffering from non-coronary microvascular disease comprising:
a) selecting a patient suffering from, or at risk of suffering from, non-coronary microvascular disease, and
b) administering to that patient an effective amount of ranolazine.

2. The method of claim 1, wherein the patient is not suffering from diabetes.

3. The method of claim 1, wherein the patient is suffering from at least one condition selected from the group consisting of hypertension, incipient diabetes, and metabolic syndrome.

4. The method of claim 1, wherein the non-coronary microvascular disease is manifested in the eye.

5. The method of claim 1, wherein the non-coronary microvascular disease is manifested in the brain.

6. The method of claim 1, wherein the non-coronary microvascular disease is manifested in the kidney.

7. The method of claim 1, wherein the non-coronary microvascular disease is manifested in the liver.

8. The method of claim 1, wherein the non-coronary microvascular disease is manifested as Restless Legs Syndrome.

9. The method of claim 1, wherein the ranolazine is administered to the patient as an oral dose.

10. The method of claim 9, where in the oral dose is a sustained release tablet.

11. The method of claim 9, wherein the sustained release tablet comprises at least 50% by weight ranolazine, a pH dependent binder, and a pH independent binder.

12. The method of claim 11, wherein the sustained release table comprises at least 50% by weight ranolazine, from about 5 to about 12.5% by weight methacrylic acid copolymer, from about 1 to about 3% by weight of hydroxypropyl methylcellulose, microcrystalline cellulose, sodium hydroxide, and magnesium stearate.

13. A method for reducing non-cardiovascular ischemia in a patient at risk of non-cardiovascular ischemia which method comprises administering ranolazine to said patient.

14. The method of claim 13, wherein said ischemia is selected from the group consisting of cerebral ischemia, renal ischemia, and ischemia associated with organ transplant.
